(19) United States
(12) Patent Application Publication
(10) Pub. No.: US 2004/0202703 A1
(43) Pub. Date: Oct. 14, 2004

(54) Antimicrobial wound covering article

(75) Inventors: Wolfgang Meyer-Ingold, Hamburg (DE); Holger Kartheus, Hamburg (DE); Michael Schink, Hamburg (DE); Michael Bogdahn, Buxtehude-Neukloster (DE); Jurgen Christian Quandt, Klein Nordende (DE); Christian Gade, Neu Wulmstorf (DE); Thorsten Berg, Hamburg (DE); Jan Janichen, Hamburg (DE)

Correspondence Address: GREENBLUM & BERNSTEIN, P.L.C., 1950 ROLAND CLARKE PLACE, RESTON, VA, 20191, US

(73) Assignee: BEJERSDORF AG, Hamburg (DE)

(21) Appl. No.: 10/681,236

(22) Filed: Oct. 9, 2003

(30) Foreign Application Priority Data
Apr. 9, 2003 (DE) 103 16 156.2

(51) Int. Cl. A61L 15/00; A61K 33/38

(52) U.S. Cl. 424/445; 424/618

(57) ABSTRACT

An antimicrobial polymeric composite for use in wound management products. The composite comprises a polymeric material which is suitable for prolonged direct contact with a wound. At least a part of the polymeric material is associated with a glass which comprises an antimicrobial element M, preferably silver. This abstract is neither intended to define the invention disclosed in this specification nor intended to limit the scope of the invention in any way.

### CROSS-REFERENCE TO RELATED APPLICATIONS

The present application claims priority under 35 U.S.C. §119 of German Patent Application No. 103 16 156.2, filed on Apr. 9, 2003, the disclosure of which is expressly incorporated by reference herein in its entirety.

### BACKGROUND OF THE INVENTION

1. Field of the Invention

The present invention relates to polymeric composites for use in wound management products. In particular, the invention relates to composite materials which comprise one or more polymeric materials which are suitable for prolonged direct contact with a wound, and a glass which comprises an element M which shows antimicrobial activity. The glass, when coming into contact with an aqueous liquid, is capable of releasing ions of said element M into the aqueous liquid.

2. Discussion of Background Information

The treatment and healing of bacterially contaminated and/or infected skin and wounds is a great challenge to medicine and the natural sciences. Poorly healing wounds and chronic wounds in particular are often populated by a wide variety of microorganisms which greatly delay or sometimes even prevent entirely the course of healing. Even with acute wounds, however, caused by trauma, surgical intervention, or even just simple injury, the penetration of pathogenic microorganisms cannot be ruled out in every case.

As a result of the penetration of germs, the wound is colonized with microorganisms. A wound populated with more than 10⁵ CFU/g is referred to as an infected wound (M. C. Robson “Clinical Research can improve the outcome of treatment of problem wounds: Infection as a Paradigm”, 8th Annual Meeting of the ETRS, Copenhagen, DK, Aug. 27-30, 1998). The massive colonization of the wound medium with microorganisms may result in a massive interference with the course of healing, which may lead ultimately to mortality. Frequent causative organisms of bacterial wound infections belong to the genera Pseudomonas, Staphylococcus, Clostridium and, among the yeasts and molds, to the genera Candida and Aspergillus. Limitation to a few species is impossible, since many of the microorganisms may be regarded as opportunistic pathogens.

Various possibilities are described for removing microorganisms from the contaminated or infected tissue of a wound and/or for killing them therein. In addition to the oral administration of antibiotics, the removal of pathogenic microorganisms from a wound may be achieved, in accordance with the prior art, by the topical application of a disinfectant or an antibiotic. However, antiseptics and antibiotics are cytotoxic, and, moreover, many pathogenic strains have developed resistances to antibiotics. The fact that the development of resistance even to an antiseptic is possible has been reported for triclosan-resistant E. coli bacteria (McMurry, L. M. et al., FEMS Microbiol Lett. 1998, 166(2): 305-9, Cookson, B. D. et al., Lancet 1991, 337 (8756): 1548-9; Uhl, S., Lancet 1993, 342(8865): 248). The principal critical factor in that case was the widespread and prophylactic use of triclosan (Irgasan®) in soaps, deodorants, textiles and plastics.

A well-known use, for example, for the antimicrobial and/or preventive therapy of contaminated or infected wounds is that of oxidants (for example iodine tincture) or antiseptics (for example, ointments containing silver sulfadiazine). Another form in which such agents are used is that of correspondingly antimicrobially coated or impregnated wound coverings and dressing materials. The use of zeolites containing silver is also known in the field of wound management products. DE-A1-19958458 provides an overview of the known antimicrobially active dressing materials containing silver. DE-A1-19958458 discloses wound coverings which comprise a synthetic polymer material that contains zeolites having metal ions therein. Also known, from EP-A1-1116698 and EP-A1-1116700, are silver containing glasses that show antimicrobial activity. However, these are embedded in thermoplastic polymers that are used only in the household and sanitary sectors in a variety of forms, such as wallpaper, cutting boards, and the like.

The development of antimicrobial wound dressings focuses not only on antimicrobial activity, but also on the creation of a moist wound environment, which—in contrast to dry wound treatment such as, e.g., with gauze compresses—provides physiological, and hence better, conditions for the natural processes of wound healing.

EP-A1-1159972 discloses a wound dressing consisting of a self-adhesive hydrocolloid composition that permits a moist wound environment and contains an antimicrobial agent which comprises silver, copper, and zinc.

An antibacterial hydrocolloid dressing is available on the market from Coloplast company under the trade name Contreet-H®. Due to an impregnation with ionic silver, this dressing permits moist wound treatment and antibacterial activity. Corresponding dressings are described in WO 00/09173 and U.S. Pat. No. 5,681,575, as well as in WO 02/062403 and WO 02/078755. WO 02/062403 describes an antimicrobial wound dressing that contains, in an adhesive matrix, a silver containing complex which comprises at least one element of Group IV of the Periodic Table of Elements. Titanium, hafnium and zirconium are mentioned as preferred elements, and the complex preferably is a phosphate complex. The silver ions, present in an amount of 0.01 to 30 mg/cm² of wound dressing, are released only on contact with an ionic solution. In this context, only adhesive materials that do not reduce the release of the silver or the antimicrobial activity of the silver are suitable. As an example, a polyurethane foam is described as a matrix, wherein the employment of a foam is a critical factor with respect to the release rate.

WO 02/078755 describes an antimicrobial wound dressing showing a release of silver of from 50 to 10,000 μg/cm² and an absorption capacity for wound exudate of more than 0.09 g/cm². Like in the case of WO 02/062403, the silver compounds are present in the form of complexes with elements of Group IV of the Periodic Table. A zirconium phosphate complex is preferred here as well. The very wide range of release rates specified in WO 02/078755 encompasses release rates that are also achieved with the silver containing antimicrobial wound dressings of the prior art.

The entire disclosures of the documents discussed above, as well as those of all other documents mentioned in the present specification, are expressly incorporated by reference herein as if the disclosure of each of these documents in its entirety were part of the present specification.

All of the above-described known antimicrobial wound dressings which contain silver have at least one problem in common, however. They all exhibit dark discoloration after a certain period due to the formation of elemental silver or silver oxide. This dark discoloration is accelerated by heat, moisture, light and/or the influence of radiation. Moreover, the unappealing dark brown or black silver oxide no longer has any germicidal effect, with the result that the efficacy of the known dressing materials suffers with regard to durability. The antimicrobial effect diminishes after a short time unless expensive sealing and packaging is employed for such products. In particular, these products do not satisfy the aesthetic demands of consumers.

It would be advantageous to have available an antimicrobial polymer material which, when used as a dressing material, especially as a wound covering, combines all of the desirable characteristics of the known individual antimicrobial dressing materials while at the same time avoiding the disadvantages of these materials.

In particular, the polymer material desirably should satisfy all or most of the following requirements:

preferably be self-adhesive to obviate the need for additional attachment materials,

have a simple structure so that it can easily be handled even by laypeople,

generate a moist wound environment where desirable,

show antimicrobial activity, yet with a minimum content of active substance,

store well without sacrificing any of the aforementioned properties,

satisfy the aesthetic demands of consumers,

be stable under external influences such as light, moisture and/or radiation and, in particular, show no discoloration even after having been exposed to these external influences for a relatively long time.

### SUMMARY OF THE INVENTION

The present invention provides an antimicrobial polymeric composite for use in wound management products. The composite comprises one or more polymeric materials which are suitable for prolonged direct contact with a wound. At least a part of said at least one or more polymeric materials is associated with a glass. The glass comprises an element M which exhibits antimicrobial activity and is selected from Ag, Au, Pd, Pt, Cu, Ir, Zn, Sn, Sb and/or Bi. When coming into contact with an aqueous liquid, the glass is capable of releasing ions of said element M into the aqueous liquid. The glass comprises about 30-60 mole-% of P₂O₅, about 20-55 mole-% of of CaO and/or MgO, up to about 5 mole-% of Na₂O, K₂O and/or Li₂O, and about 5-50 mole-% of SiO₂, Al₂O₃ and/or B₂O₃.

In one aspect of the composite of the present invention, the one or more polymeric materials may be selected form polyacrylates, styrene butadiene block copolymers, natural rubbers, polysaccharides, heteropolysaccharides and combinations thereof.

In another aspect, the one or more polymeric materials may comprise a polyurethane, preferably, a polyurethane comprising units which are derived from a polyether polyol.

In yet another aspect, the one or more polymeric materials may comprise a hydrocolloid. The hydrocolloid may be selected, for example, from of microcrystalline cellulose, cellulose, alkylcellulose, hydroxyalkylcellulose, hydroxyalkylalkylcellulose and/or carboxyalkylcellulose and salts thereof.

In a still further aspect, the one or more polymeric materials may comprise agar agar, carrageen, tragacanth, gum arabic, alginate, pectin, guar flour, carob flour, starch, dextrin, gelatin, xanthan, chitosan and/or casein.

In another aspect, the one or more polymeric materials may comprise a hydrogel.

In another aspect, the one or more polymeric materials may contain a pigment, e.g., a white pigment. The pigment which may, for example, comprise TiO₂ is preferably present in an amount of from about 0.01% by weight to about 2% by weight, based on the weight of the one or more polymeric materials.

In a further aspect, the one or more polymeric materials may comprise a superabsorber, preferably in an amount of from about 0.5% by weight to about 30% by weight, based on the weight of the one or more polymeric materials.

In yet another aspect of the composite, the one or more polymeric materials may contain one or more species which are independently selected from elemental Al, Zn and Mg and basic compounds of these elements. If employed, the one or more species preferably are present in an amount of from about 0.01% by weight to about 5% by weight, based on the weight of the one or more polymeric materials.

In a still further aspect of the composite of the present invention, the glass may comprise about 40-60 mole-% of P₂O₅, about 35-55 mole-% of CaO and/or MgO, up to about 5 mole-% of Na₂O, K₂O and/or Li₂O, and about 5-20 mole-% of SiO₂ and/or Al₂O₃, and may contain from about 0.1% by weight to about 10% by weight of Ag₂O, ZnO and/or CuO. By way of non-limiting example, the glass may comprise about 45-55 mole-% of P₂O₅, about 35-50 mole-% of CaO and/or MgO, up to about 5 mole-% of Na₂O and/or K₂O, and about 5-15 mole-% of SiO₂ and/or Al₂O₃, containing from about 0.5% by weight to about 8% by weight of Ag₂O and/or ZnO.

In another aspect, the glass may comprise about 45-55 mole-% of P₂O₅, about 40-50 mole-% of MgO and about 5-10 mole-% of Al₂O₃, and may contain from about 1% by weight to about 3% by weight of Ag₂O. Preferably, silver is the only element M contained in the glass and is present therein in an amount of from about 1.5% by weight to about 2.5% by weight of Ag₂O.

In another aspect of the composite of the present invention, the glass may be present in an amount of from about 0.01% by weight to about 40% by weight, e.g., in an amount of from about 0.05% by weight to about 10% by weight, or in an amount of from about 0.1% by weight to about 5% by weight, based on the combined weight of the glass and the one or more polymeric materials.

In yet another aspect, the glass may comprise not more than about 5% by weight of residual water and/or the glass may comprise a particulate glass. For example, the glass may comprise particles which have a volume-related particle size of from about 0.1 μm to about 10 μm.

In another aspect, at least a part of the one or more polymeric materials may form a matrix which comprises at least a part of the particulate glass. Still further, the one or more polymeric materials may be gel-like materials and/or may have a liquid absorption capacity of at least about 0.5 g/g.

In a still further aspect of the composite of the invention, the composite preferably is capable of releasing silver ions for at least about 24 hours upon being contacted with a wound exudate.

In another aspect of the composite, at least a part of the one or more polymeric materials may form a sheet-like structure. At least a part of the sheet-like structure may be foamed.

In another aspect, at least a part of the one or more polymeric materials and of the glass may form a layer which has an area weight of from about 400 g/m² to about 1200 g/m² and/or may form a sheet-like layer having a thickness of from about 0.1 mm to about 2 mm. The sheet-like structure (layer) may have a water vapor permeability of from about 100 g/(m²*24 h) to about 5,000 g/(M²*24 h), for example, a water vapor permeability of from about 300 g/(m 2*24 h) to about 1,500 g/(m²*24 h).

In a still further aspect of the composite of the present invention, at least a part of the composite may comprise a sheet-like structure and the composite further may comprise a backing layer. Preferably, the backing layer comprises a polymeric material. For example, the polymeric material may comprise a polyurethane and/or a polyolefin. Furthermore, the backing layer may comprise a pressure sensitive adhesive on one side thereof.

The present invention also provides a wound management article which comprises the composite of the present invention, including the various aspects thereof, as discussed above. The wound management article may comprise, for example, a bandage, a wound dressing, a wound pad or a compress. Preferably, the article releases the element M (preferably silver) at a rate of from about 5 mg/(m²*24 h) to about 50 mg/(m²*24 h), for example, at a rate of from about 10 mg/(m²*24 h) to about 40 mg/(m²*24 h).

The present invention also provides a method of covering a wound. According to this method, the polymeric composite of the present invention, including the various aspects thereof, as discussed above, is placed in direct contact with the wound.

Furthermore, the present invention provides a process for producing the polymeric composite of the present invention, including the various aspects thereof, as discussed above. According to this process, a particulate glass is combined with one or more polymeric materials which are suitable for prolonged direct contact with a wound so that at least a part of the one or more polymeric materials forms a matrix which has at least a part of the particulate glass incorporated therein. The glass comprises about 30-60 mole-% of P₂O₅, about 20-55 mole-% of CaO and/or MgO, up to about 5 mole-% of Na₂O, K₂O and/or Li₂O, and about 5-50 mole-% of SiO₂, Al₂O₃ and/or B₂O₃ and contains an element M which comprises at least one of Ag, Cu, and Zn. This glass, when coming into contact with an aqueous liquid, is capable of releasing ions of said element M into the aqueous liquid.

In one aspect, this process may further comprise a sterilization of the composite, e.g, by irradiation of the composite with γ-rays.

The present invention also provides a polymeric composite for use in wound management products, wherein the composite comprises one or more polymeric materials which are suitable for prolonged direct contact with a wound and are selected from polyacrylates, polyurethanes, styrene butadiene block copolymers, natural rubbers, polysaccharides, heteropolysaccharides and polypeptides. At least a part of said one or more polymeric materials is associated with a glass which comprises an element M which shows antimicrobial activity and is selected from Ag, Au, Pd, Pt, Cu, Ir, Zn, Sn, Sb and/or Bi. This glass, when coming into contact with an aqueous liquid, is capable of releasing ions of said element M into the aqueous liquid. The glass is present in an amount of from about 0.01% by weight to about 40% by weight, based on the combined weight of the glass and the one or more polymeric materials.

In one aspect, the one or more polymeric materials may comprise a polyurethane, a natural rubber, a polysaccharide and/or a heteropolysaccharide. In another aspect, they may comprise a hydrocolloid. The hydrocolloid may, for example, comprise microcrystalline cellulose, cellulose, alkylcellulose, hydroxyalkylcellulose, hydroxyalkylalkylcellulose and/or carboxyalkylcellulose and salts thereof.

In yet another aspect, the one or more polymeric materials may comprise agar agar, carrageen, tragacanth, gum arabic, alginate, pectin, guar flour, carob flour, starch, dextrin, gelatin, xanthan, chitosan and/or casein. In a still further aspect, the one or more polymeric materials may comprise a hydrogel.

In yet another aspect of the composite, the one or more polymeric materials may have a white pigment incorporated therein, e.g., in an amount of from about 0.01% by weight to about 2% by weight, based on the weight of the one or more polymeric materials.

In a further aspect, the one or more polymeric materials may comprise a superabsorber in an amount of from about 0.5% by weight to about 30% by weight, based on the weight of the one or more polymeric materials. The superabsorber may, for example, have units which are derived from acrylic acid.

In a still further aspect, the one or more polymeric materials may contain therein one or more species independently selected from elemental Al, Zn and Mg and basic compounds thereof, in an amount of from about 0.01% by weight to about 5% by weight, based on the weight of the one or more polymeric materials.

In another aspect of the composite, the glass may be present in an amount of from about 0.05% by weight to about 10% by weight, e.g., in an amount of from about 0.1% by weight to about 5% by weight, based on the combined weight of the glass and the one or more polymeric materials. The glass preferably comprises not more than about 5% by weight of residual water. In yet another aspect, the glass preferably comprises a particulate glass. For example, the glass may comprise particles having a volume-related particle size of from about 0.1 lm to about 10 μm.

In a still further aspect of the composite of the present invention, the glass may comprise about 40-60 mole-% of P₂O₅, about 35-55 mole-% of CaO and/or MgO, up to about 5 mole-% of Na₂O, K₂O and/or Li₂O, and about 5-20 mole-% of SiO₂ and/or Al₂O₃, and may contain from about 0.1% by weight to about 10% by weight of Ag₂O, ZnO and/or CuO.

For example, the glass may comprise about 45-55 mole-% of P₂O₅, about 35-50 mole-% of CaO and/or MgO, up to about 5 mole-% of Na₂O and/or K₂O, and about 5-15 mole-% of SiO₂ and/or Al₂O₃, and may contain about 0.5% by weight to about 8% by weight of at least one of Ag₂O and ZnO. Preferably, the glass comprises about 45-55 mole-% of P₂O₅, about 40-50 mole-% of MgO, and about 5-10 mole-% of Al₂O₃, and contains about 1% by weight to about 3% by weight of Ag₂O. In another aspect, silver may be the only element M contained in the glass and may be present therein in an amount of about 1.5% by weight to about 2.5% by weight of Ag₂O.

The composite preferably is capable of releasing silver ions for at least about 48 hours when in contact with a wound exudate.

In another aspect of the composite, at least a part of the one or more polymeric materials forms a matrix which comprises at least a part of the glass and/or at least a part of the one or more polymeric materials forms a sheet-like structure. In the latter case, at least a part of the sheet-like structure may be foamed and/or may have a thickness of from about 0.1 mm to about 2 mm. Preferably, the sheet-like structure has a water vapor permeability of from about 100 g/(m²*24 h) to about 5,000 g/(m²*24 h), in particular, a water vapor permeability of from about 300 g/(m²*24 h) to about 1,500 g/(m²*24 h).

In a still further aspect of the composite, at least a part of the one or more polymeric materials and of the glass may form a layer which has an area weight of from about 400 g/m² to about 1200 g/m².

In yet another aspect, the one or more polymeric materials may have a liquid absorption capacity of at least about 1.5 g/g.

In another aspect, the composite may further comprises a backing layer. This backing layer preferably comprises a polymeric material such as, e.g., a polyurethane and/or a polyolefin. Also, the backing layer may have a pressure sensitive adhesive on one side thereof.

The present invention also provides a wound management article which comprises the composite of the present invention, including the various aspects thereof, as discussed above. The wound management article may comprise, for example, a bandage, a wound dressing, a wound pad or a compress. Preferably, the article releases the element M (preferably silver) at a rate of from about 10 mg/(m²*24 h) to about 40 mg/(m²*24 h).

The present invention also provides a method of covering a wound. According to this method, at least a portion of the polymeric composite of the present invention, including the various aspects thereof, as discussed above, is placed in direct contact with the wound.

Furthermore, the present invention provides a process for producing the polymeric composite of the present invention, including the various aspects thereof, as discussed above. According to this process, one or more polymeric materials which are suitable for prolonged direct contact with a wound and are selected from polyacrylates, polyurethanes, styrene butadiene block copolymers, natural rubbers, polysaccharides, heteropolysaccharides and polypeptides are combined with a glass. The glass contains an element M which comprises Ag, Cu and/or Zn, and, when coming into contact with an aqueous liquid, the glass is capable of releasing ions of said element M into the aqueous liquid. The glass is employed in an amount of from about 0.1% by weight to about 10% by weight, based on the combined weight of the glass and the one or more polymeric materials.

In one aspect, this process may further comprise a sterilization of the composite, e.g, by irradiation of the composite with γ-rays. Preferably, this sterilization does not result in a noticeable discoloration of the glass.

The polymeric composite according to the present invention has antimicrobial properties and comprises a (substantially biocompatible) polymeric material. Non-limiting examples of the polymeric material are materials commonly used in wound healing, such as synthetic polymer materials, for example, polyurethanes, polyacrylates, SIBS (styrene-isobutylene-styrene) block copolymers, SEBS (styrene-ethylene butylene-styrene) block copolymers, natural rubbers and also chitosans, alginates, hydrogels, hydrocolloids and the like. Particularly preferred as polymeric materials are polyurethanes. It is not absolutely necessary for the polymeric material to be foamed, as is required in the prior art for effective release of the antimicrobial element M, particularly, silver. In this regard, in the description of the present invention which follows, silver will for reasons of convenience, frequently be referred to as representative of the antimicrobial element M. It is to be understood, however, that the reference to silver, which is a preferred element M for use in the present invention, is meant to include a reference to any element M, or any combinations thereof.

Preferably, self-adhesive polymeric materials are chosen in order to avoid the need for additional adhesive on the edge of the composite of the present invention. Particularly preferred according to the present invention is the use of self-adhesive polyurethane resins that may be used as a hydroactive wound covering for moist wound healing. By way of non-limiting example, elastic, cross-linked polyurethanes as described, for example, in WO 97/43328 A1 may be used for this purpose. An area weight of a corresponding polyurethane structure may be, for example, in the range of from about 50 g/m² to about 2500 g/m².

Generally, polyurethanes are prepared from the known starting materials of polyurethane chemistry according to known methods. Examples of such methods are described, for example, in the following documents: DE-OS 3103499, DE-OS 3103500, EP0 147588A1, EP0665 856B1 and DE 196 18825A1.

A conventional process for synthesizing a polyurethane (c) is the reaction of an alcohol (a) with an isocyanate (b), as illustrated in the following reaction scheme:

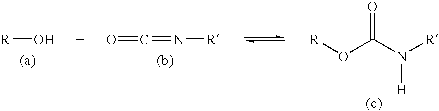

A particular advantage of polyurethane polymers or gels is their self-adhesive properties, which obviates the need for additional application of an adhesive layer to the polymeric material to affix the latter to the skin. For example, a polyurethane matrix which contains a silver containing glass may be sandwiched between a cover layer that is firmly anchored thereto, also known as the backing layer, and a removable release layer. The removable release layer serves to secure the adhesive layer on the backing layer, to improve transport and storage stability, and is removed before application to the skin.

Non-limiting examples of suitable polyurethanes for use in a matrix as discussed above are described in, e.g., DE 196 18 825, which document discloses hydrophilic, self-adhesive polyurethane gels which are made by using

(a) polyether polyols having 2 to 6 hydroxyl groups, OH numbers of from 20 to 112, and an ethylene oxide (EO) content of ≧10% by weight,

(b) antioxidants,

(c) bismuth(III) carboxylates based on carboxylic acids having 2 to 18 carbon atoms as polymerization catalysts, which carboxylates are soluble in the polyols (a), and

(d) hexamethylene diisocyanate.

In these polyurethanes, the product of the functionalities of the polyurethane-forming components (a) and (d) is at least about 5.2, the amount of catalyst (c) ranges from about 0.005 to about 0.25% by weight based on the polyol (a), the amount of antioxidant (b) is in the range of from about 0.1 to about 1.0% by weight based on polyol (a). Furthermore, the ratio of the free NCO groups of component (d) and the free OH groups of component (a) (isocyanate index) is in the range of from about 0.30 to about 0.70.

When polyether polyols are employed as polyol components for the preparation of polyurethanes, those having 3 to 4, more preferably 4, hydroxyl groups, and an OH number of from about 20 to about 112, more preferably, of from about 30 to about 56, are preferably used. The ethylene oxide content of the polyether polyols preferably is ≧ about 20% by weight.

Polyether polyols for use in the preparation of polyurethanes are known per se and may be prepared, for example, by polymerizing epoxides such as, e.g., ethylene oxide, propylene oxide, butylene oxide or tetrahydrofuran, with themselves, or by subjecting these epoxides, preferably ethylene oxide and propylene oxide, optionally as a mixture with one another or separately in succession, to an addition reaction with starter components containing at least two reactive hydrogen atoms such as, e.g., water, ethylene glycol, propylene glycol, diethylene glycol, dipropylene glycol, glycerol, trimethylol propane, pentaerythritol, sorbitol and sucrose. Further examples of polyhydroxy compounds which may be used are disclosed, for example, in High Polymers, Vol. XVI, “Polyurethanes, Chemistry and Technology” (Saunders-Frisch, Interscience Publishers, New York, Vol. 1, 1962, pp. 32-42).

Preferred examples of polyisocyanates for use in the synthesis of polyurethanes include monomeric and/or trimerized hexamethylene diisocyanate, and hexamethylene diisocyanate which may be modified by biuret, uretdione or allophanate groups, and/or may be prepolymerized with polyether polyols or mixtures of polyether polyols. These polyether polyols, in turn, are preferably based on the conventional starter compounds which contain 2 or more reactive hydrogen atoms and the conventional epoxides, such as ethylene oxide or propylene oxide, e.g, those having an OH number of ≦850, preferably of from about 100 to about 600. Particularly preferred is the use of modified hexamethylene diisocyanate, in particular, hexamethylene diisocyanate modified by prepolymerization with polyether diols having an OH number of from about 200 to about 600. Preferably, the content of residual monomeric hexamethylene diisocyanate in these products is below about 0.5% by weight.

Preferred examples of catalysts for the preparation of polyurethane gels for use in the present invention are bismuth(III) carboxylates based on linear, branched, saturated or unsaturated carboxylic acids having about 2 to about 18 carbon atoms, preferably about 6 to about 18 carbon atoms, which carboxylates are soluble in the above anhydrous polyether polyols (a). Bi(III) salts of branched, saturated carboxylic acids with tertiary carboxyl groups, such as 2,2-dimethyloctanoic acid (for example, Versatic acids, Shell) are particularly preferred. Compositions of these Bi(III) salts in excess amounts of these carboxylic acids are frequently used. A solution of 1 mole of the Bi(III) salt of the Versatic 10 acid (2,2-dimethyloctanoic acid) in an excess of about 3 mole of this acid with a Bi content of about 17% has proven outstanding.

It is preferred to use the catalysts in quantities of from about 0.03 to about 0.25%, in particular, to about 0.1% by weight, based on the polyol component (a).

Non-limiting examples of antioxidants which may be contained in the polyurethane gels and other polymeric materials for use in the present invention comprise, in particular, sterically hindered phenolic stabilizers, such as BHT (2,6-di-tert-butyl-4-methylphenol), Vulkanox BKF (2,2′-methylene-bis-(6-tert-butyl-4-methylphenol) (Bayer AG, Germany), Irganox 1010 (pentaerythrityl tetrakis[3-(3,5-di-tert-butyl-4-hydroxyphenyl)propionate]), Irganox 1076 (octadecyl 3-(3,5-di-tert-butyl-4-hydroxyphenyl)propionate) (Ciba-Geigy, Switzerland) and tocopherol (Vitamin E). The use of antioxidants of the α-tocopherol type is particularly preferred. The antioxidants are preferably used in amounts of from about 0.15 to about 0.5% by weight based on the polyol component (a).

The isocyanate index (ratio of the free NCO groups and the free OH groups used in the reaction) of polyurethane gel compositions suitable for use in the present invention generally is in the range of from about 0.30 to about 0.70, preferably in the range from about 0.45 to about 0.60, depending to some extent on the functionality of the polyisocyanate and polyol components employed. The isocyanate index necessary for gel formation can be estimated by using the following formula:

f_{(polyol)}·(f_{(isocyanate)}−1)·index≈2

$$index \approx \frac{2}{{f}_{( polyol )} • ( {f}_{( isocyanate )} - 1 )}$$

f: functionality of the polyisocyanate or polyol component

Depending on the desired tackiness or elasticity of the gel, the isocyanate index for affording optimum results may deviate from the calculated value by up to ±about 20%. The polyurethane gel compositions for use in accordance with the present invention may be prepared by conventional processes, as described, for example, in Becker/Braun, Kunststoff-Handbuch, Vol. 7, Polyurethane, p. 121 ff., Carl-Hauser, 1983.

Additional preferred examples of polyurethane gel compositions for use in the present invention are disclosed in EP 0 665 856 A. According to this document, polyurethane gel compositions are obtainable from

1. a polyurethane gel which comprises

(A) 25-62% by weight, preferably 30-60% by weight, particularly preferably 40-57% by weight, based on the total of (A) and (B) of a covalently cross-linked polyurethane as high molecular weight matrix and

(B) 75-38% by weight, preferably 70-40% by weight, particularly preferably 60-43% by weight, based on the total of (A) and (B), of one or more polyhydroxy compounds which are firmly bonded to the matrix by secondary valence forces and have an average molecular weight between 1,000 and 12,000, preferably between 1,500 and 8,000, particularly preferably between 2,000 and 6,000, and an average OH number between 20 and 112, preferably between 25 and 84, particularly preferably between 28 and 56, as liquid dispersant, the dispersant being essentially free of hydroxy compounds with a molecular weight below 800, preferably below 1,000, particularly preferably below 1,500 and, optionally,

(C) 0 to 100% by weight, based on the total of (A) and (B), of fillers and/or additives,

which gel composition is obtainable by reacting a mixture of

a) one or more polyisocyanates,

b) one or more polyhydroxy compounds with an average molecular weight between 1,000 and 12,000, and with an average OH number between 20 and 112,

c) optionally, catalysts or accelerators for the reaction between isocyanate groups and hydroxyl groups and, optionally,

d) fillers and additives,

this mixture being essentially free of hydroxyl compounds with a molecular weight below 800, the average functionality of the polyisocyanates (F₁) being between 2 and 4, the average functionality of the polyhydroxy compounds (F_{P}) being between 3 and 6, and the isocyanate index (K) being represented by the formula

$$K = \frac{300 \pm X}{( {F}_{I} • {F}_{P} ) - 1} + 7$$

in which X≦120, preferably X≦100, particularly preferably X≦90, and the index K has values between 15 and 70, where the stated averages of molecular weight and OH number are to be understood as number averages,

2. a water-absorbing material and/or

3. a non-aqueous foaming agent.

When a self-adhesive polyurethane is to be produced, the conditions defined above should be adhered to in selecting the gel-forming components, since otherwise non-adhesive, elastic gels rather than self-adhesive gels may be obtained.

Preferred polyhydroxy compounds are polyether polyols, as are listed in detail in the above-mentioned applications.

Suitable as polyisocyanate components for use in the synthesis of the above polyurethane gels are both (cyclo)aliphatic and aromatic polyisocyanates. Preferred (cyclo)aliphatic polyisocyanates are 1,6-hexamethylene diisocyanate and its biurets and trimers, and hydrogenated diphenylmethane diisocyanate (“MDI”) types. Preferred aromatic polyisocyanates are those obtained by distillation, such as mixtures of the 4,4′- and 2,4′-isomers of MDI, 4,4′-MDI, and toluylene diisocyanate (“TDI”) types.

The polyisocyanates may be chosen, for example, from unmodified aromatic or aliphatic diisocyanates or else, from modified products thereof as formed by prepolymerization with amines, polyols, including polyether polyols and the like.

Polyurethanes have several properties which make them particularly suitable for use in the present invention:

Polyurethanes can be prepared both as self-adhesive and non-adhesive polymers, as desired.

When self-adhesive polyurethane systems are employed, the addition of adhesives that under certain circumstances may induce side effects such as maceration, inflammation of the dermal areas, reduced cutaneous respiration, etc. can be dispensed with.

Self-adhesive polyurethanes are advantageous as compared to other adhesive materials, such as polyacrylates, rubber, etc., since they have no allergy potential.

Polyurethanes have very good permeability to water vapor. As a result, the release of water through the skin does not cause maceration in the case of application for a relatively long time period.

The oxygen permeability of polyurethanes ensures the necessary oxygen supply to the covered skin area, thus preventing damage to the tissue. Polyurethanes are hypoallergenic, so no allergic reactions will occur after application.

In contrast to other materials such as many hydrocolloids and hydrogels, polyurethanes exhibit no tendency to disintegrate when in relatively long contact with fluids such as wound exudate. Consequently, a polyurethane wound dressing, for example, that is left in contact with wound fluids for a relatively long period, leaves no residues in the wound that hinder wound healing.

Self-adhesive polyurethanes lose their adhesiveness on contact with liquid, thereby avoiding sticking to freshly formed tissue and, moreover, permitting painless removal of a corresponding wound covering.

Polyurethane wound coverings in accordance with the present invention generate a moist wound environment, which leads to faster wound healing.

Other polymeric materials such as, e.g., hydrocolloids, may be used in the present invention instead of, or in addition to, polyurethane materials. The hydrocolloids for use in cosmetic and pharmaceutical products comprise the following preferred classes:

organic, natural materials, such as, e.g., agar agar, carrageen, tragacanth, gum arabic, alginates, pectins, polysaccharides, guar flour, carob flour, starches, dextrins, gelatins and casein;

organic, modified natural materials such as, e.g., carboxymethylcellulose and other cellulose ethers, hydroxyethylcellulose, hydroxypropyl cellulose, microcrystalline cellulose and the like;

organic, fully synthetic materials such as, e.g., polyacrylic and polymethacrylic compounds, vinyl polymers, polycarboxylic acids, polyethers, polyimines, polyamides, polyurethanes,

inorganic materials, for example, polysilicic acids, clay minerals such as montmorillonites, zeolites and silicas.

Microcrystalline cellulose is a preferred example of a hydrocolloid for use as a polymeric material in accordance with the present invention. A preferred representative is available, for example, from “FMC Corporation Food and Pharmaceutical Products” under the trade name Avicel®. A particularly advantageous product for use in the present invention is Avicel® RC 591, which is modified microcrystalline cellulose composed of 89% microcrystalline cellulose and 11% sodium carboxymethyl cellulose. Additional commercial products in this class of materials include Avicel® RC/CL, Avicel® CE 15, and Avicel® 500.

Further examples of advantageous hydrocolloids for use in the present invention include methyl ethers of cellulose, also called methylcelluloses. Methylcelluloses may be represented by the general formula:

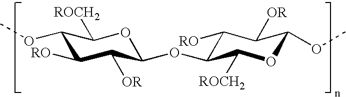

where R represent a hydrogen atom or a methyl group.

Particularly advantageous for use as polymeric material in accordance with the present invention are the mixed cellulose ethers, likewise generally known as methylcelluloses, which contain 2-hydroxyethyl groups, 2-hydroxypropyl groups and/or 2-hydroxybutyl groups in addition to a predominant content of methyl groups. Particularly preferred are (hydroxypropyl)methyl celluloses, such as those available under the trade name Methocel® E4M from Dow Chemical Corp.

Further examples of advantageous polymeric materials for use in the present invention include sodium carboxymethylcellulose, i.e., the sodium salt of the glycolic acid ether of cellulose, for which R in the above general formula represents a hydrogen atom and/or CH₂—COONa. Particularly preferred are the sodium carboxymethylcelluloses which are available from Aqualon under the trade name Natrosol Plus 330 CS, and which are also known as cellulose gum.

Another preferred example of a polymeric material for use in the present invention is xanthan (CAS No. 11138-66-2), also called xanthan gum, which is an anionic heteropolysaccharide that generally is made by fermentation from corn sugar and is isolated as the potassium salt. It is produced by Xanthomonas campestris and a few other species under aerobic conditions. Its molecular weight usually ranges from 2×10⁶ to 24×10⁶. Xanthan features a main chain formed by β-1,4 bonded glucose units (cellulose) and side chains. The side chains comprise units of glucose, mannose, glucuronic acid, acetate and pyruvate. Xanthan is the designation of the first anionic heteropolysaccharide of microbial origin. It is produced by Xanthomonas campestris and a few other species under aerobic conditions with a molecular weight of 2-15×10⁵. The number of pyruvate units determines the viscosity of the xanthan. Xanthan may be produced in two-day batch cultures with a yield of 70-90%, based on the carbohydrate employed. Yields of 25-30 g/l are achieved. Processing takes place after killing the culture by precipitation with, for example, 2-propanol. Xanthan is subsequently dried and powdered.

Another example of an advantageous polymeric material for use in the present invention is carrageen, a gel-forming agent with a structure similar to that of agar. Carageen constitutes an extract from North Atlantic red algae belonging to the genus florideans (Chondrus crispus and Gigartina stellata).

The designation carrageen is frequently used for the dried algae product, and the designation carrageenan is used for the extract therefrom. The carrageen precipitated out of the hot water extract of the algae is a colorless to sand-colored powder with a molecular weight ranging from 100,000-800,000 and a sulfate content of approximately 25%. Carrageen is very readily soluble in warm water; upon cooling, a thixotropic gel forms even at a water content of 95-98%. The firmness of the gel results from the double helix structure of the carrageen. Three principal constituents are distinguished in carrageenan: the gel-forming K fraction consists of D-galactose-4-sulfate and 3,6-anhydro-α-D-galactose which are bonded together by glycosidic bonds alternating in the 1,3- and 1,4-positions (agar, by contrast, contains 3,6-anhydro-α-L-galactose). The non-gelling k fraction is composed of 1,3-glycosidically bonded D-galactose-2-sulfate and 1,4-bonded D-galactose-2,6-disulfate radicals, and is readily soluble in cold water. The ιcarrageenan, consisting of 1,3-bonded D-galactose-4-sulfate and 1,4-bonded 3,6 anhydro-α-D-galactose-2-sulfate, is both water-soluble and gel-forming. Further carrageen types are likewise identified by Greek letters: α, β, γ, μ, ν, ξ, π, ω, χ. The type of cations present (K⁺, NH₄⁺, Na⁺, Mg²⁺, Ca²⁺) also affects the solubility of the carrageens.

The use of chitosan, yet another preferred example of a polymeric material suitable for use in the present invention, in cosmetic preparations is known per se. Chitosan is a partially deacylated chitin. This biopolymer has film-forming properties and is characterized by a silky feel on the skin. As is known, chitosan is used, for example, in hair care. It is suitable, more so than the chitin on which it is based, as a thickener or stabilizer and improves the adhesion and water resistance of polymeric films. Exemplary for the large number of literature references which discuss chitosan is H. P. Fiedler, “Lexikon der Hilfsstoffe für Pharmazie, Kosmetik und angrenzende Gebiete”, 3rd Edition 1989, Editio Cantor, Aulendorf, p. 293, keyword “Chitosan”.

Chitosan may be represented by the general formula:

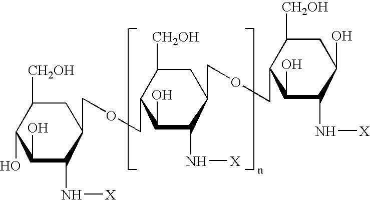

where n can assume values up to about 10,000, and X represents acetyl or hydrogen.

Chitosan is formed by the deacetylation and partial depolymerization (hydrolysis) of chitin, which may be represented by the general formula:

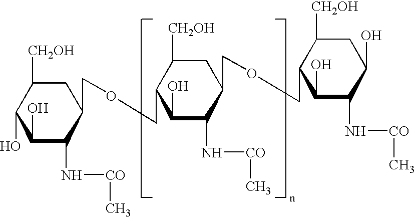

Chitin is an essential constituent of the exoskeleton of arthropods (e.g. insects, crabs, spiders) and may also be found in supporting tissues of other organisms (e.g. mollusks, algae and fungi).

At pH<6, chitosan is positively charged and in that pH range, it is soluble in aqueous systems. It is incompatible with anionic raw materials. Therefore, for the preparation of chitosan-containing oil-in-water emulsions, the use of nonionic emulsifiers is appropriate. Such emulsifiers are known per se, for example from EP 0 776 657 A1.

Preferred chitosans for use in the present invention are chitosans having a degree of deacetylation of >25%, in particular >55 to 99% (determined by ¹H-NMR).

It may also be advantageous to employ chitosans having molecular weights between about 10,000 and about 1,000,000, in particular those having molecular weights between about 100,000 and about 1,000,000 (determined by means of gel permeation chromatography).

Polyacrylates are further examples of advantageous polymeric materials for use in the present invention. Preferred polyacrylates include acrylate-alkyl acrylate copolymers, in particular those from the group of so-called carbomers or Carbopols (Carbopol® is a registered trademark of the B.F. Goodrich Company). Specifically, these acrylate-alkyl acrylate copolymers may be represented by the general formula:

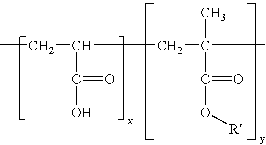

where R′ represents a long-chain alkyl radical and x and y are numbers representing the stoichiometric ratios of the corresponding comonomer units.

Particularly preferred in accordance with the present invention are acrylate copolymers and/or acrylate-alkyl acrylate copolymers available from the B.F. Goodrich Company under the trade names Carbopol® 1382, Carbopol® 981 and Carbopol® 5984; other preferred polyacrylates from the Carbopol group are the types 980 and 2984. A particularly preferred carbomer is Carbomer 2001.

Copolymers of C₁₀₋₃₀ alkyl acrylates and one or more monomers selected from acrylic acid, methacrylic acid and esters thereof, cross-linked with an allyl ether of saccharose and/or an allyl ether of pentaerythritol, are also advantageous polymeric materials for use in the present invention.

Compounds known under the INCI name “Acrylates/C₁₀₋₃₀ Alkyl Acrylate Crosspolymer” are advantageous as well. Particularly preferred are those available under the trade names Pemulen TR1 and Pemulen TR2 from the B.F. Goodrich Company.

Compounds known under the INCI name ammonium acryloyldimethyltaurate/VP copolymers are also preferred for use in the present invention. Particularly advantageous are ammonium acryloyldimethyltaurate/VP copolymers of the empirical formula [C₇H₁₆N₂SO₄]ₙ[C₆H₈NO]ₘ, and a structure of the general formula:

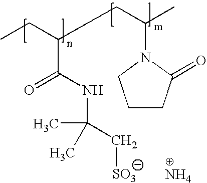

Particularly preferred materials in this regard have the Chemical Abstracts registration numbers 58374-69-9, 13162-05-5 and 88-12-0, and are available, e.g., under the trade name Aristoflex® AVC from Clariant GmbH, Germany.

Also advantageous as polymeric material for use in the present invention are copolymers/crosspolymers which comprise acryloyldimethyl taurate, such as Simugel® EG, available from, e.g., the company Seppio S.A., Italy.

The polymeric material, particularly a polyurethane material, used in the present invention may be partially or fully foamed or substantially unfoamed. Foaming of the polymeric material results in a softer matrix system, which provides a pleasant texture for the user and makes it possible to manufacture an article such as a dressing that conforms better to the body. Moreover, foamed wound coverings provide a cushioning effect, which may be desirable in pressure-sensitive applications such as burn injuries.

However, it may also be advantageous to use a polymeric material which is substantially unfoamed. Such a material will still have excellent properties. According to the present invention, the release of the silver does not depend on the foam structure.

The polymeric material for use in the present invention may contain fillers and other additives. Non-limiting examples thereof are inorganic or organic short fibers, superabsorbers, pigments such as, e.g., titanium dioxide, zinc oxide and metal pigments, plasticizers, colorants, surface-active agents, and liquid extenders such as, e.g., substances with a boiling point above 150° C., etc.

Examples of inorganic fillers include barite, calcium carbonate, gypsum, kieserite, soda, cerium dioxide, silica sand, kaolin, carbon black and microbeads.

Non-limiting examples of organic fillers that may be used include powders on the basis of polystyrene, polyvinyl chloride, urea formaldehyde resin and polyhydrazodicarbonamide.

Non-limiting examples of short fibers that may be used include, for example, glass fibers about 0.1-1 mm in length and fibers of organic origin, for example polyester or polyamide fibers.

Metal powders, such as iron, aluminum or copper powders, may be used as well.

The addition of a pigment such as, e.g., titanium dioxide, preferably in an amount of about 0.01 to about 2% by weight based on the polymeric material, improves the aesthetic appeal of a wound covering material according to the present invention in that the user cannot see any unaesthetic blood through a dressing, for example.

Organic and inorganic colorants and/or pigments may be used according to the present invention to impart a desired color to the polymeric material. Non-limiting examples thereof are iron oxide and chromic oxide pigments, and phthalocyanine and monoazo based pigments.

Cellulose powder, activated charcoal and silicic acid may be mentioned as non-limiting examples of surface-active substances for use in the present invention.

Adding coloring agents and the like to the polymeric material is not mandatory, since no discoloration of the silver glass containing polymeric materials will take place. Rather, coloring with additional substances will usually serve to individualize the finished product, for example to make it more attractive for children or to adapt it to specific environmental parameters such as skin color.

A superabsorber (preferably in the form of a powder) may be incorporated into the polymeric material for use in the present invention, e.g., for holding (particularly large amounts of) fluid. The superabsorber helps absorb the fluid that is released from the skin area, thus countering maceration and keeping a wound covering material from coming off prematurely. In addition, in the case of open wounds, the increased retention of secretions from the wound and the increased absorption of pathogenic microorganisms associated therewith affords a significant advantage.

Preferred water-absorbing materials for use as superabsorbers are water-absorbing salts of polyacrylates and copolymers thereof, in particular the sodium or potassium salts. These products are commercially available, and may be non-crosslinked or crosslinked. Particularly preferred products are products disclosed in DE 37 13 601 A1, and superabsorbers of the new generation with a low residual water content, which can be removed, and a high swelling capacity under pressure. Specific examples of these preferred products include polymers based on acrylic acid/sodium acrylate with a small amount of crosslinking. Sodium polyacrylates of this type can be obtained under the trade name Favor 22-SK (Stockhausen & Co. KG, Germany). Other superabsorbers are suitable as well, for example, carboxymethylcellulose and karaya.

It is advantageous to incorporate the superabsorber or superabsorbing polymer into the polymeric material in an amount of from about 0.01% to about 40% by weight, particularly from about 0.5% to about 30% by weight, more particularly about 20% by weight, based on the total weight of the polymeric material.

According to preferred embodiment of the present invention, elemental aluminum, zinc and/or magnesium and/or basic compounds thereof such as, e.g., zinc hydroxide or magnesium chloride, may be added to the polymeric material. The addition of these substances (preferably in anhydrous form) may, for example, take place during the synthesis of the polymeric material and/or the incorporation of the silver glass.

These substances may increase the quantity of the antimicrobial (silver) glass that can be combined with the polymeric material and/or they may permit the addition of further antimicrobial compounds, such as silver zeolites. Additionally, the addition of elemental aluminum, zinc and/or magnesium and/or the basic compounds thereof may prevent the undesirable black discoloration of the conventional silver compounds, if present.

Particularly, the moisture from the wound or from the environment causes a conversion of the silver to silver chloride or silver oxide, which can cause known silver containing wound covering materials to look unappealing and to lose efficacy. The addition of aluminum, zinc or magnesium allows regeneration of the antimicrobially active silver in accordance with the electrochemical potentials of the reactions: 2 AgCl+Zn, 2/3 Al, Mg→2 Ag+ZnCl₂, MgCl₂, 2/3 AlCl₃

In accordance with the present invention, the elemental Al, Zn, Mg and/or the basic compounds thereof, are preferably employed in a (total) amount of from about 0.01 to about 5% by weight, based on the total weight of the polymeric material.

To modify the adhesion characteristics of a given polymeric material, vinyl polymers, polyacrylates and other polymers and copolymers which are suitable for use as adhesives, as well as adhesives based on natural substances, may be added to the polymeric material, usually in a concentration of up to about 10% by weight, based on the weight of the polymeric material, without diluting or impairing the advantageous properties of the polymeric material, in particular, of the polyurethane materials.

The polymeric composite of the present invention comprises a glass which contains at least one element M which shows antimicrobial activity. Preferably, the element M comprises at least silver.

The term “antimicrobial” as used in the present specification and the appended claims is to be understood in its broadest sense, and is inclusive of terms like “disinfectant”, “antibacterial”, “antifungal” etc. In particular, “antimicrobial” denotes activity against pathogenic microorganisms of any kind.

In addition to the antimicrobial element M, the glass for use in the present invention will usually comprise at least P₂O₅; CaO and/or MgO; and one or more of Al₂O₃, SiO₂, and B₂O₃ as glass forming elements. Often it will also comprise one or more of Na₂O, K₂O and Li₂O, as well as one or more other elements which are frequently present in glass (e.g., metals, including transition metals and rare earth elements, for example, in order to change the color of the glass or the melting point thereof, etc.). However, if one or more other elements are present, the total amount thereof will usually not exceed about 5 mole-%, particularly not exceed about 2 mole-%, e.g., not exceed about 1 mole-%, calculated as oxides and based on the total glass composition. Preferably, the glass is substantially colorless and/or transparent. This is even more preferred in cases where the remainder of the composite is substantially colorless and/or transparent as well.

In this regard, it is to be understood that while the glass forming elements are represented herein in the form of their oxides, this is merely to follow the convention in the art, and should by no means construed to indicate that these elements must be present as oxides. Rather, the elements can be present in any form, e.g., as salts, complexes and the like. By the same token, the mole and weight percentages given herein are calculated based on the assumption that all of the indicated elements are present in the oxide form, even if in reality some of them are not. Again, this calculation corresponds to the convention in the glass field. Furthermore, unless indicated otherwise, all mole and weight percentages of the glass components given herein are based on the totality of all elements present in the glass, each element being represented and calculated as oxide.

P₂O₅ will often be a major component of the glass for use in the present invention. It will usually be present in an amount of at least about 30 mole-%, e.g., at least about 35 mole-%, at least about 40 mole-%, or even at least about 45 mole-%, and usually in an amount of not more than about 75 mole-%, e.g., not more than about 70 mole-%, not more than about 65 mole-%, not more than about 60 mole-%, not more than about 55 mole-%, or even not more than about 50 mole-%.

One or both of CaO and MgO will usually be present in the glass for use in the present invention in a total amount of at least about 20 mole-%, e.g., at least about 25 mole-%, at least about 30 mole-%, or even at least about 35 mole-%, and usually in a total amount of not more than about 55 mole-%, e.g., not more than about 50 mole-%, or even not more than about 45 mole-%. Preferably, the glass for use in the present invention contains at least MgO.

One or more of Al₂O₃, SiO₂, and B₂O₃ will usually be present in the glass for use in the present invention in a total amount of at least about 5 mole-%, e.g., or even at least about 10 mole-%, and usually in a total amount of not more than about 50 mole-%, e.g., not more than about 40 mole-%, not more than about 30 mole-%, not more than about 20 mole-%, or even not more than about 15 mole-%. Preferably, the glass for use in the present invention contains both Al₂O₃ and SiO₂, or at least Al₂O₃ alone. Al₂O₃ will usually be present in an amount of at least about 5 mole-%, although usually not more than about 10 mole-%. The amounts of SiO₂, when present, can be as high as about 50 mole-%, particularly in cases where the content of P₂O₅ is at the lower end of the indicated range.

One or more of Na₂O, K₂O and Li₂O will often be present in the glass for use in the present invention in a total amount of not more about 5 mole-%, e.g., not more than about 3 mole-%, or even not more than about 2 mole-%. More frequently, the glass for use in the present invention will contain one or both of Na₂O and K₂O, or at least Na₂O alone.

The glass for use in the present invention will include one or more antimicrobial elements M in a (total) amount, expressed as oxide(s), of usually at least about 0.1% by weight, e.g., at least about 0.2% by weight, at least about 0.5% by weight, at least about 0.8% by weight, or even at least about 1% by weight, although the content of these oxides will usually not exceed about 10% by weight, e.g., not exceed about 8% by weight, not exceed about 5% by weight, not exceed about 3% by weight, or even not exceed about 2.5% by weight. Usually, the glass will contain as element M at least one of Ag, Cu and Zn, preferably at least one of Ag and Zn, most preferably at least Ag. If silver is present, it will often be present in an amount of at least about 0.1 mole-%, e.g., at least about 0.2 mole-%, at least about 0.3 mole-%, or at least about 0.5 mole-%, but usually not more than about 8 mole-%, e.g., not more than about 5 mole-%, not more than about 2.5 mole-%, or even not more than about 1 mole-%.

In this regard, a particularly preferred glass for use in the present invention has the following approximate composition:

| | % by weight |
|---|---|
| P₂O₅ | about 73.5 |
| MgO | about 18.5 |
| Al₂O₅ | about 6.5 |
| Ag₂O | about 2 |

Corresponding antimicrobial glasses are available from a variety of commercial sources, for example, Ishizuka Glass Co. Ltd., Japan, and Mo-Sci Corporation, U.S.A.

The polymeric composite of the present invention will usually contain the glass which comprises the antimicrobial element M in an amount, based on the total weight of this glass and the one or more polymeric materials (without fillers, additives etc.), of at least about 0.01% by weight, e.g., at least about 0.05% by weight, at least about 0.075%, or at least about 0.01% by weight. Usually, however, the amount of this glass will be not higher than about 40% by weight, e.g., not higher than about 20% by weight, not higher than about 10% by weight, not higher than about 5% by weight, not higher than about 2% by weight, or not higher than about 1% by weight. Of course, two or more different kinds of silver containing glass can be used in combination according to the present invention. By way of non-limiting example, two different kinds of antimicrobial element M containing glass may be used, e.g., a first glass which releases M at a faster rate and/or releases M in a higher or lower amount than a second glass used in combination therewith. Additionally, the different kinds of glass may contain different elements M.

Preferably, the antimicrobial glass will have a residual moisture content of not more than about 5% by weight, e.g., not more than about 2.5% by weight.

According to the present invention, at least a part (and preferably substantially all) of the antimicrobial glass will be present in particulate form although it is to be understood that the glass may also be employed in any different form that permits the use of the composite of the present invention in wound management products. By way of non-limiting example, the glass may be present in the form of a thin film etc.

Any desired shape of the particulate glass may be employed, e.g., (short) fibers, granules, (micro)beads, microspheres, flakes, etc. However, the particle size should be small enough to not significantly distract from the intended use of the article. Preferably, the volume-related particle size should not be higher than about 10 μm, e.g., not higher than about 5 μm, although no significant advantage may be obtained by decreasing the particle size to less than about 0.1 μm, or even less than about 1 μm. The glass may have been made by a variety of methods, for example, by casting (including centrifugal casting) and by rod, fiber and tube drawing. Other methods include glass foaming, pressing and comminution.

In general, the amount of the antimicrobial glass will be selected to provide an amount of element M, especially when (only) silver is present as element M, expressed as mg of element M per kg of the polymeric composite which comprises the glass, the one or more polymeric materials and optionally employed additives, fillers etc, of at least about 5 mg/kg, e.g., at least about 10 mg/kg, at least about 20 mg/kg, or at least about 30 mg/kg, and usually not more about 200 mg/kg, e.g., not more about 150 mg/kg, not more about 100 mg/kg, or not more about 50 mg/kg.

Furthermore, it is preferred for the composite of the present invention to release the element M, especially silver, at a rate of at least about 5 mg, more preferably at least about 10 mg, per kg of composite, as determined by the method described below (i.e., over a period of 24 hours). Usually, the release rate will not be higher than about 100 mg/kg, e.g., not higher than about 50 mg/kg, or even not higher than about 30 mg/kg. The release rate can be controlled through the quantity and composition of the antimicrobial glass, and also, inter alia, through the choice and amount of optional additives.

The release of antimicrobially active metal M ions (especially Ag ions) from the polymeric composite in accordance with the present invention (as determined by the test described below) will usually take place for a period of at least about 2 hours, e.g., at least about 10 hours, at least about 24 hours, at least about 96 hours, or even at least 240 hours, so that a long-term treatment of the wound is ensured. This obviates the need for a frequent changing of the wound management product, e.g., a wound dressing.

The antimicrobial glass may be present in the composite of the present invention in any form that permits at least a part of the glass to be associated with, preferably, in direct contact with at least a part of the one or more polymeric materials. According to a preferred embodiment, the one or more polymeric materials form a matrix in which the (preferably) particulate glass is embedded. However, the glass may also be present in one or more different forms, e.g., in the form of a glass layer, or a layer which comprises the (particulate) glass, which is in contact with one or more layers formed by the one or more polymeric materials, e.g., sandwiched between two layers or on the surface of a single layer. Combinations of different forms of contact between glass and polymeric materials are possible as well.

The manner in which the glass and the one or more polymeric materials are combined with each other is apparently dependent, inter alia, on the desired structure of the polymeric composite. By way of non-limiting example, the polymeric material(s) which may be present in molten form (e.g., in an extruder) or dissolved in a suitable solvent, and the particulate glass may be mixed and the resultant mixture may then be given the desired shape, optionally after at least a part of the solvent has been removed. Alternatively, a solution or dispersion of the polymeric material(s) in a solvent which also contains the (particulate) antimicrobial glass may be applied (by spraying, casting, extrusion, etc.) on a shaped body or layer formed by the same or a different polymeric material which may optionally already contain the same or a different antimicrobial glass. Alternatively, the glass may be added before or during the reaction which affords the desired polymeric materials.

For example, the (particulate) glass may be added to a reaction mixture comprising a polyol and a polyisocyanate, resulting in a highly homogeneous dispersion of the glass in the resulting polyurethane. It was found that the antimicrobial (e.g., silver containing) glass used in accordance with the present invention may be incorporated into a polymer matrix, and in particular, into a polyurethane matrix, by admixing the glass with the polymer starting materials, without interfering with the polymerization reaction. Of course, any other form of bringing the glass and the polymeric material into (permanent) contact with each other may be used as well.

It was found that the glass usually does not give rise to any significant degradation of the properties of the polymer matrix. Also, the glass usually will not be subject to discoloration (as a result of being exposed to, e.g., radiation and/or heat), even if the wound management product is used for extended periods of time.

The antimicrobial composite of the present invention may be employed in many different forms and shapes. A preferred composite of the present invention has a sheet-like structure (foamed or not foamed), with a uniform or non-uniform thickness. The thickness of the composite in this case will usually be at least about 100 μm, e.g., at least about 400 μm, or at least about 600 μm, but will usually be not higher than about 2,000 μm, e.g., not higher than about 1,500 μm, or not higher than about 1,200 μm.

Also, a sheet-like composite of the present invention will usually have a water vapor permeability, determined according to the method described below, of at least about 100 g/(m²*24 h), preferably at least about 250 g/(m²*24 h), or even at least about 300 g/(m²*24 h). On the other hand, the water vapor permeability will usually not exceed 5,000 g/(m²*24 h), e.g., not exceed about 2,500 g/(m²*24 h), or not exceed 1,500 g/(m²*24 h).

The water absorption capacity of the one or more polymeric materials for use in the composite of the present invention, determined by the method described below, usually will be at least about 0.5 g water per g of polymeric material, preferably at least about 1 g/g, or even at least about 1.5 g/g. It will usually be not higher than about 10 g/g, e.g., not higher than about 6 g/g, or not higher than about 3.5 g/g.

If the composite in accordance with the present invention is made to be self-adhesive, additional means of attaching the composite to the skin may be dispensed with. The composite may be placed directly on the wound to be covered, e.g., as a bandaging material, and will adhere to the skin surrounding the wound due to its self-adhesive properties.

In the case of relatively large wounds and/or if additional adhesion is desired, or if the composite is not made to be self-adhesive, the composite may be adhered to the skin by the addition of an adhesive for the edge portion. A corresponding wound management article in accordance with the present invention will have a structure similar to that of known products, e.g., known bandages. In this case, the wound management article will generally comprise a backing material that is provided on one side with a self-adhesive coating. The composite in accordance with the invention is then affixed to this self-adhesive coating. In order to ensure ease of use, the self-adhesive coating may further be covered with a protective layer, such as a sealing paper.

A non-limiting example of a suitable adhesive for use as the surface adhesive (=adhesive for the edge portion) which may be used in combination with the optional backing material is described in DE 27 43 979 C3. Alternatively and preferably, commercial pressure-sensitive acrylate-based or rubber-based adhesives may be used for the adhesive coating.

Particularly preferred surface adhesives are thermoplastic hot-melt adhesives based on natural and synthetic rubbers and other synthetic polymers such as acrylates, methacrylates, polyurethanes, polyolefins, polyvinyl derivatives, polyester or silicone, optionally with suitable additives such as tackifying resins, plasticizers, stabilizers, and other auxiliary materials as needed. In some cases, secondary cross-linking by UV or electron beam radiation may be of advantage.

Hot-melt adhesives based on block copolymers, in particular, are characterized by their wide versatility, since a controlled reduction of the glass transition temperature of the self-adhesive substance resulting from the choice of tackifier, plasticizers, molecule size and the molecular weight distribution of the starting components ensures the required bonding to the skin in a manner appropriate to their function, even at critical points of the human locomotor system.

A high shear strength of the hot-melt adhesive may be achieved through a high cohesiveness of the polymer. The good finger tack results from the range of tackifiers and plasticizers used. The adhesive composition preferably contains at least one aromatic component, which usually accounts for less than about 35%, preferably from about 5% to about 30% by weight, of the adhesive. For systems which are to adhere particularly strongly, the hot-melt adhesive may preferably be based on block copolymers, especially A-B or A-B-A block copolymers or mixtures thereof. The hard phase A comprises primarily polystyrene or its derivatives and the soft phase B comprises ethylene, propylene, butylene, butadiene, isoprene or mixtures thereof, particular preference being given to ethylene and butylene or mixtures thereof. The controlled blending of diblock and triblock copolymers is particularly advantageous, with a diblock copolymer fraction of less than about 80% by weight being preferred.

In a preferred embodiment the hot-melt adhesive has the composition indicated below:

| | |
|---|---|
| about 10% b. w. to about 90% b. w. | block copolymers, |
| about 5% b. w. to about 80% b. w. | tackifiers such as, e.g., oils, waxes, |
| | resins and/or mixtures thereof, |
| | preferably mixtures of |
| | resins and oils, |
| less than about 60% b. w. | plasticizers, |
| less than about 15% b. w. | additives, |
| less than about 5% b. w. | stabilizers. |

The aliphatic or aromatic oils, waxes and resins which are preferably used as tackifiers usually are hydrocarbon oils, waxes and resins, with the consistency of the oils, such as paraffinic hydrocarbon oils, or the waxes, such as paraffinic hydrocarbon waxes, being responsible for their favorable effect on bonding to the skin. The plasticizers will usually be selected from medium- or long-chain fatty acids and/or their esters. The plasticizers serve to adjust the adhesion properties and the stability. If desired, further stabilizers and other auxiliaries may be employed.

The carrier (backing) material for use in a wound management article according to the present invention preferably comprises an air and water vapor permeable, but water-impermeable, polymeric layer. Usually, this layer will have a thickness of from about 10 μm to about 100 μm. The backing material, which under certain circumstances may be flexible, preferably comprises polymers selected from at least one of polyurethanes, polyethylene, polypropylene, polyamides, polyesters and polyether esters. However, other known backing materials such as, e.g., cloth, non-woven fabrics, foams, plastics, etc. may also be used.

The polymeric composite in accordance with the present invention may be affixed to the backing layer or film in a known manner. The polymeric composite having the backing material on one side thereof may be applied to the skin as a composite sheet with the backing layer on top. The permeability to water vapor, the strength of the wound covering, the cushioning for pressure, as well as other physical properties of the wound covering may be controlled by the selection of the backing material.

When a polymeric composite according to the present invention comes into contact with a wound exudate, it will usually absorb (at least a part of) the exudate, causing the polymeric material(s) to swell. A particularly satisfactory absorption of wound exudate by the polymeric composite is observed when polyurethane materials are used, especially in combination with superabsorber materials.

Two important steps for wound healing can subsequently be identified. Firstly, silver ions are released by the (preferably finely distributed) glass in contact with the exudate, and secondly, pathogens from the wound can be taken up by the polymeric material(s). After application to an exuding wound, a silver glass containing composite in accordance with the present invention will, through contact between the fluid and the silver glass, kill the pathogens in the wound fluid and/or prevent colonization of the wound by microorganisms and possibly an infection. These two steps individually, and both of them synergistically, bring about a reduction in pathogen growth and/or the death of the pathogens. The antimicrobial polymeric composite in accordance with the present invention thus will possess both bacteriostatic and bactericidal properties. When the composite is removed, the antibacterial effect ceases. There is no need to wash the wound to remove, for example, previously applied antiseptics and antibiotics.

The invention described is thus based on the antimicrobial effect of antimicrobial metal containing glass in combination with a (preferably highly) absorbent polymeric material, which together achieve a synergistic effect. Moreover, a wound covering such as, e.g., a polyurethane wound covering in accordance with the present invention, can have self-adhesive properties that allow it to be affixed to the intact skin on the edge of the patient's wound. Moreover, the present invention provides a new type of wound covering that can be used to treat infected wounds, or for preventive care against wound infections. The covering constitutes a barrier to microorganisms, preventing them from penetrating from outside, in that they are killed on contact with the antimicrobial wound covering.

The combination of a polymer such as, e.g., a hydroactive polyurethane polymeric material with a silver containing glass results in advantageous product appeal for consumers in addition to great product stability. In particular, the unappealing dark discoloration exhibited by known silver containing materials, which may be unacceptable to the consumer, and which is caused in particular by moisture, light or y rays, may be reduced significantly or even prevented entirely.

A particularly advantageous aspect of the present invention is that the antimicrobial glass can be incorporated without problems into a polymer (e.g., a polyurethane) matrix and thus makes it possible to prepare an antimicrobial wound dressing. Surprisingly, the content of antimicrobial glass in the polymer, the distribution thereof in the polymer, and also the concentrations of additives can be selected within wide ranges without sacrificing the advantages described herein.

Moreover, additional skin care and/or wound-healing ingredients that support regeneration of the skin can be incorporated into the polymer matrix. Non-limiting examples of active ingredients that may be added include vitamins such as, e.g., vitamin E or vitamin C, essential oils, flavone and its derivatives, anti-inflammatory agents, analgesics, and combinations of two or more thereof.

The antimicrobial polymeric composite in accordance with the present invention can thus be used in, for example, wound care as a wound covering that is self-adhesive or is provided with additional surface adhesive. Moreover, in addition to its use in wound healing, it may be used in other fileds such as, e.g., in skin care, as skin protection, and for the prevention of skin damage.

### BRIEF DESCRIPTION OF THE DRAWINGS

The present invention is further described in the detailed description which follows, in reference to the noted plurality of drawings by way of non-limiting examples of exemplary embodiments of the present invention, in which like reference numerals represent similar parts throughout the several views of the drawings, and wherein:

FIG. 1 is a graph which shows the results of silver release tests carried out with samples of polymeric composites according to the present invention;

FIG. 2 shows the results of discoloration tests carried out with samples of polymeric composites according to the present invention;

FIG. 3 shows the result of discoloration test carried out with a sample of a polymeric composite according to the present invention after sterilization with y rays; and

FIG. 4 shows the result of a discoloration test carried out with a sample of a polymeric composite according to the present invention after an accelerated aging thereof.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

Polymeric materials and wound covering materials in accordance with the present invention are described below. Unless indicated otherwise, relative amounts and concentrations are based on the total weight of the polymeric material.

The liquid absorption capacity and the water vapor permeability of the polymeric materials for use in the present invention referred to above are determined according to the following methods:

Determination of Liquid Absorption Capacity of Polymeric Material

A circular sample having a diameter of 2.2 cm is punched out and kept in an atmosphere of 23+/−2° C. and 50+/−5% r.h. for one hour. Then, the sample is weighed and immersed as a whole in physiological saline of 23+/−0.5° C. for 3 hours. The sample is weighed again and the liquid absorption is calculated from the difference of weights before and after immersion.

Determination of Water Vapor Permeability.

The determination was carried out according to ASTM E 96 (water method), with following modifications:

The opening of the test flask was 804 mm².

The sample was kept for 24 h at 23+/−2° C. und 50+/−5% r.h.

The distance between water level in the test flask and the sample was 35+/−5 mm.

After having been kept for 24 h at 37+/−1.5° C. and 30+/−3% r.h., the test flasks containing the samples were weighed again.

### EXAMPLE 1

The following components 1 and 2 were prepared by mixing the ingredients for 24 hours on a roller block:

Component 1:

| | |
|---|---|
| 500.0 g | Polyether Polyol (Levagel, Bayer AG, Germany)¹ |
| 1.9 g | Vitamin E (Tocopherol) |
| 51.3 g | Isocyanate Prepolymer (Desmodur, Bayer AG)² |
| 144.4 g | Superabsorber (Favor T, Degussa Stockhausen, Germany)³ |
| 1.3 g | TiO₂ |
| 1.6 g | Silver containing glass (Ionpure B1, Ishizuka Glass Company, |
| | Japan)⁴ |

Component 2:

| | |
|---|---|
| 30 g | Polyether Polyol (Levagel)¹ |
| 3 g | Bi-Catalyst⁵ |

¹Pentaerythritol/propylene oxide/ethylene oxide copolymer capped by an ethyleneoxide block; functionality: 4; OH number: 35; average molecular weight (calculated): 6,400; viscosity (23° C.): 1,000 mPas; content of ethylene oxide: 20% by weight.
²NCO-terminate prepolymer prepared by reacting, at 80° C., hexamethylene diisocyanate (HDI) and polypropylene glycol (average molecular weight: 220) in a molar ratio of 5:1 and subsequent vacuum distillation to a content of residual HDI monomer of <0.5% by weight; NCO content: 12.6% by weight; viscosity (23° C.): 5,000 mPas.
³Cross-linked sodium polyacrylate.
⁴P₂O₅ 73.35% by weight; MgO 18.33% by weight; Al₂O₃ 6.32% by weight; Ag₂O 2% by weight.
⁵Solution of 1 mol of Bi(III) salt of 2,2-dimethyloctanoic acid in 3 mol of 2,2-dimethyl octanoic acid (Bi content about 17% by weight; a corresponding product is commercially available under the trade name Coscat).

A total of 500 g of the above component 1 and 3 g of the above component 2 were mixed by hand for 40 seconds and the resultant mixture was poured onto release paper and spread thereon so as to obtain an area weight of the polyurethane composition of about 800 g/m². Curing was then carried out for 5 minutes at 65° C.

Pieces of a predetermined size were punched out of the cured polyurethane composition on the release paper to form individual wound pads. These pads were placed on the acrylate adhesive coated side of a polyurethane sheet (water vapor permeability about 350 g/(m²*24 h)) with the release paper facing away from the polyurethane sheet. The release paper was removed, the composite was covered with sealing paper and individual bandages were punched out such that the wound pads were uniformly surrounded by the acrylate coated PU sheet. The individual bandages were then sealed and sterilized (gamma radiation, 25-35 kGy).

Determination of Release of Silver

The release of silver was determined by extraction of silver from the wound dressing or the wound pad material (size: 9+/−1 cm²) into a phosphate-buffered saline solution (30 ml of PBS-solution) at 31° C. for a predetermined time (24 h). The PBS used for this method is described by Dulbecco (John Paul, ‘Zell- und Gewebekulturen’, Walter de Gruyter 1980, 92). The content of calcium and magnesium ions is adjusted to wound fluid levels (0.19 g/L of CaCl₂×2H₂O; 0.27 g/L of MgSO₄×7H₂O Geigy Scientific Tables, Vol. 3, Ciba-Geigy Ltd., 8. Ed. 1984, p. 82). Following the extraction, the sample is carefully removed and the amount of extracted silver in the aqueous phase is determined, after acidification of the sample, by atomic absorption spectroscopy at a wavelength of 328.1 nm in an air/C₂H₂ flame.

A release of 20-25 mg of silver/m² of wound covering (over 24 h) was determined.

### EXAMPLE 2

A silver glass containing polymeric composite according to the present invention was produced from the following materials:

| | |
|---|---|
| Polyether polyol (Levagel): | 16.50 g |
| Cross-linking agent (Desmodur) | 1.70 g |
| Vitamin E | 0.10 g |
| Superabsorber (Favor T) | 2.05 g |
| Silver glass (Ionpure B1) | 0.10 g |
| Bi-Catalyst | 0.04 g |
| Total | 20.50 g |

The release of Ag⁺ ions from the resultant polymeric composite was determined by the following method:

A sample A (1 g) of the composite in the form of a sheet having an area weight of about 800 g/m² was placed in 100 ml of 0.9% NaCl solution for a predetermined period of time and the amount of silver extracted from the sample was determined by atomic absorption spectroscopy. The results are shown in Table 1 below.

Comparable samples of known materials with silver zeolites (B) or silver zirconium phosphates (C), for which an antimicrobial activity had previously been demonstrated, released the silver in quantities which are also listed in Table 1.

TABLE 1
Release of Ag

| | Quantity of Ag Released (mg/kg) | | |
|---|---|---|---|
| Time (h) | A | B | C |
| 24 | 23.8 | 14.4 | 28.5 |
| 72 | 25.4 | 25.0 | 23.4 |
| 168 | 28.3 | 26.5 | 29.6 |

The results summarized in Table 1 show that the release of silver ions by the polymeric composite in accordance with the present invention (A) and by the known materials with silver zeolites (B) or silver zirconium phosphates (C) is of the same order of magnitude.

### EXAMPLE 3

Polymeric composites in accordance with the present invention having contents of silver glass different from that of the composite of Example 2 were produced from the following materials.

Sample D

| | |
|---|---|
| Polyether polyol (Levagel): | 14.505 g |
| Cross-linking agent (Desmodur) | 1.391 g |
| Vitamin E | 0.057 g |
| Superabsorber (Favor T) | 4.524 g |
| Silver glass (Ionpure B1) | 0.002 g |
| Bi-Catalyst | 0.041 g |
| Total | 20.520 g |

Sample E

| | |
|---|---|
| Polyether polyol (Levagel): | 14.41 g |
| Cross-linking agent (Desmodur) | 1.38 g |
| Vitamin E | 0.06 g |
| Superabsorber (Favor T) | 4.50 g |
| Silver glass (Ionpure B1) | 0.01 g |
| Bi-Catalyst | 0.04 g |
| Total | 20.39 g |

Sample F

| | |
|---|---|
| Polyether polyol (Levagel): | 14.41 g |
| Cross-linking agent (Desmodur) | 1.38 g |
| Vitamin E | 0.06 g |
| Superabsorber (Favor T) | 4.51 g |
| Silver glass (Ionpure B1) | 0.016 g |
| Bi-Catalyst | 0.04 g |
| Total | 20.41 g |

Sample G

| | |
|---|---|
| Polyether polyol (Levagel): | 79.03 g |
| Cross-linking agent (Desmodur) | 7.65 g |
| Vitamin E | 0.30 g |
| Superabsorber (Favor T) | 22.76 g |
| Silver glass (Ionpure B1) | 0.11 g |
| Bi-Catalyst | 0.36 g |
| Total | 110.22 g |

Sample H

| | |
|---|---|
| Polyether polyol (Levagel): | 78.68 g |
| Cross-linking agent (Desmodur) | 7.57 g |
| Vitamin E | 0.30 g |
| Superabsorber (Favor T) | 22.66 g |
| Silver glass (Ionpure B1) | 0.28 g |
| Bi-Catalyst | 0.36 g |
| Total | 109.86 g |

Sample I

| | |
|---|---|
| Polyether polyol (Levagel): | 78.95 g |
| Cross-linking agent (Desmodur) | 7.58 g |
| Vitamin E | 0.31 g |
| Superabsorber (Favor T) | 22.74 g |
| Silver glass (Ionpure) | 0.57 g |
| Bi-Catalyst | 0.36 g |
| Total | 110.51 g |

Sample J

| | |
|---|---|
| Polyether polyol (Levagel): | 79.16 g |
| Cross-linking agent (Desmodur) | 7.55 g |
| Vitamin E | 0.30 g |
| Superabsorber (Favor T) | 22.91 g |
| Silver glass (Ionpure B1) | 1.14 g |
| Bi-Catalyst | 0.36 g |
| Total | 111.42 g |

### EXAMPLE 4

Samples D-J (area weight about 800 g/m²) from Example 3 were tested with regard to their silver ion release after 24 h using the following method.

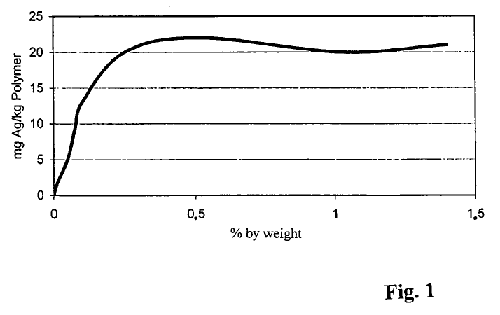

A sample (30 cm²) was placed in 100 ml isotonic saline solution at 32° C. After 24 h the sample was removed and the solution was filtered through a 0.45 μm membrane filter, whereafter the concentration of the silver in the solution was determined using graphite tube AAS. The table below and FIG. 1 summarize the results.

| | % by weight of silver glass | Release of Ag |
|---|---|---|
| Sample | in sample | (mg Ag/kg sample) |
| D | 0.01 | 1.6 |
| E | 0.05 | 5.2 |
| F | 0.075 | 9.4 |
| G | 0.1 | 13.0 |
| H | 0.25 | 20.0 |
| I | 0.52 | 22.0 |
| J | 1.02 | 20.0 |

### EXAMPLE 5

Samples G and I were tested for antimicrobial activity according to JIS 2801:2000 for

Escherichia coli IFO 3972

Staphylococcus aureus IFO 12732.

The antimicrobial activity of the samples was calculated according to equation (1) below:

$$Antimicrobial activity = {log}_{10} \frac{Number of living bacteria at beginning}{Number of living bacteria after 24 h} \qquad ( 1 )$$

Antimicrobial activity may be assumed at a value of >2, which means that the number of tested bacteria was reduced by a factor of >100.

As shown in Table 2, all tested samples possess adequate antimicrobial activity.

TABLE 2
Antimicrobial Activity

| | | Antimicrobial | |
|---|---|---|---|
| | | Activity per Equation (1) | |
| | Ag Release after 24 h | Escherichia | |
| Sample | [mg/mg] | coli | Staphylococcus aureus |
| G | 13 | >3.6 | >3.3 |
| I | 22 | >3.6 | >3.3 |
| I sterile | 20 | >3.6 | >3.3 |

### EXAMPLE 6

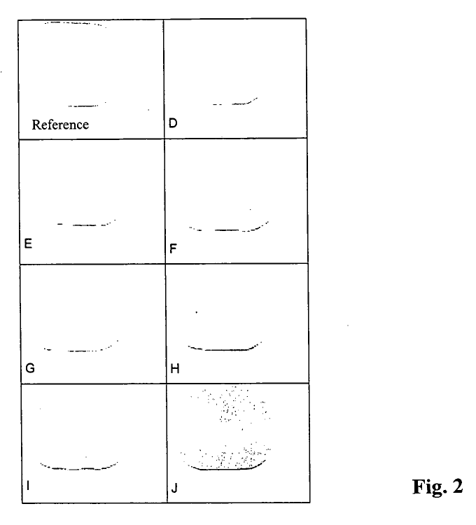

To compare the stability against discoloration of the composites in accordance with the present invention, samples D through J from Example 3 were tested for their color change caused by the addition of the silver glass. FIG. 2 shows samples D through J as a black and white copy. No change in color as compared to the undoped reference sample is visible up to sample H. Only at a concentration above 0.25% by weight of silver glass (sample H) can a slight color change be discerned that is difficult to see with the naked eye.

### EXAMPLE 7

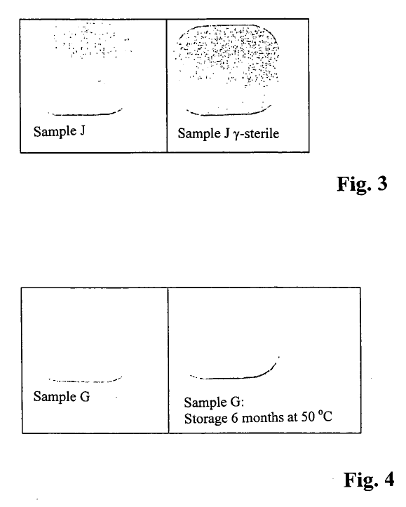

To test the stability of the silver glass during sterilization, sample J from Example 3 was γ-sterilized with 26 kGy. As can be seen from FIG. 3, the γ-sterilization does not produce any color change. A γ-sterilization of the finished composite did not cause any loss in antimicrobial activity per JIS Z 2801:2000 and, surprisingly, also did not cause any discoloration of the composite.

### EXAMPLE 8

To test stability with regard to aging of the composites in accordance with the present invention, sample G from Example 3 above was subjected to accelerated aging for 6 months at 50° C. and evaluated with regard to color stability. Here, too, no color changes were observed, as can clearly be seen even from the b/w copies reproduced in FIG. 4. This shows that the composite of the present invention shows high discoloration stability under regular and even more severe conditions. However, it is noted that under extremely harsh conditions, i.e., prolonged exposure to bright sunlight, a discoloration of composites according to the present invention has occasionally been observed.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. An antimicrobial polymeric composite for use in wound management products, wherein the composite comprises one or more polymeric materials which are suitable for prolonged direct contact with a wound, at least a part of said at least one or more polymeric materials being associated with a glass which comprises an element M which exhibits antimicrobial activity and comprises at least one of Ag, Au, Pd, Pt, Cu, Ir, Zn, Sn, Sb and Bi, which glass, when coming into contact with an aqueous liquid, is capable of releasing ions of said element M into the aqueous liquid and comprises about 30-60 mole-% of P₂O₅, about 20-55 mole-% of at least one of CaO and MgO, up to about 5 mole-% of at least one of Na₂O, K₂O and Li₂O, and about 5-50 mole-% of at least one of SiO₂, Al₂O₃ and B₂O₃.

2. The composite of claim 1, wherein the one or more polymeric materials comprise at least one of a polyacrylate, a styrene butadiene block copolymer, a natural rubber, a polysaccharide, and a heteropolysaccharide.

3. The composite of claim 1, wherein the one or more polymeric materials comprise a polyurethane.

4. The composite of claim 3, wherein the polyurethane comprises units which are derived from a polyether polyol.

5. The composite of claim 1, wherein the one or more polymeric materials comprise a hydrocolloid.

6. The composite of claim 5, wherein the hydrocolloid comprises at least one of microcrystalline cellulose, cellulose, alkylcellulose, hydroxyalkylcellulose, hydroxyalkylalkylcellulose and carboxyalkylcellulose and salts thereof.

7. The composite of claim 1, wherein the one or more polymeric materials comprise at least one of agar agar, carrageen, tragacanth, gum arabic, alginate, pectin, guar flour, carob flour, starch, dextrin, gelatin, xanthan, chitosan and casein.

8. The composite of claim 1, wherein the one or more polymeric materials comprise a hydrogel.

9. The composite of claim 1, wherein the one or more polymeric materials have a pigment incorporated therein.

10. The composite of claim 9, wherein the pigment comprises TiO₂.

11. The composite of claim 9, wherein the pigment is present in an amount of from about 0.01% by weight to about 2% by weight, based on the weight of the one or more polymeric materials.

12. The composite of claim 1, wherein the one or more polymeric materials comprise a superabsorber.

13. The composite of claim 12, wherein the superabsorber is present in an amount of from about 0.5% by weight to about 30% by weight, based on the weight of the one or more polymeric materials.

14. The composite of claim 1, wherein the one or more polymeric materials have incorporated therein a species which comprises at least one of elemental Al, Zn and Mg and a basic compound thereof.

15. The composite of claim 14, wherein the at least one species is present in an amount of from about 0.01% by weight to about 5% by weight, based on the combined weight of the one or more polymeric materials.

16. The composite of claim 1, wherein the glass comprises about 40-60 mole-% of P₂O₅, about 35-55 mole-% of at least one of CaO and MgO, up to about 5 mole-% of at least one of Na₂O, K₂O and Li₂O, and about 5-20 mole-% of at least one of SiO₂ and Al₂O₃, and contains from about 0.1% by weight to about 10% by weight of at least one of Ag₂O, ZnO and CuO.

17. The composite of claim 16, wherein the glass comprises about 45-55 mole-% of P₂O₅, about 35-50 mole-% of at least one of CaO and MgO, up to about 5 mole-% of at least one of Na₂O and K₂O, and about 5-15 mole-% of at least one of SiO₂ and Al₂O₃, and contains from about 0.5% by weight to about 8% by weight of at least one of Ag₂O and ZnO.

18. The composite of claim 1, wherein the glass comprises about 45-55 mole-% of P₂O₅, about 40-50 mole-% of MgO, and about 5-10 mole-% of Al₂O₃, and contains from about 1% by weight to about 3% by weight of Ag₂O.

19. The composite of claim 18, wherein silver is the only element M contained in the glass and is present in an amount of from about 1.5% by weight to about 2.5% by weight of Ag₂O.

20. The composite of claim 1, wherein the glass is present in an amount of from about 0.01% by weight to about 40% by weight, based on the combined weight of the glass and the one or more polymeric materials.

21. The composite of claim 17, wherein the glass is present in an amount of from about 0.05% by weight to about 10% by weight, based on the combined weight of the glass and the one or more polymeric materials.

22. The article of claim 18, wherein the glass is present in an amount of from about 0.1% by weight to about 5% by weight, based on the combined weight of the glass and the one or more polymeric materials.

23. The composite of claim 1, wherein the glass comprises not more than about 5% by weight of residual water.

24. The composite of claim 17, wherein the composite is capable of releasing silver ions for at least about 24 hours upon being contacted with a wound exudate.

25. The composite of claim 1, wherein the glass comprises a particulate glass.

26. The composite of claim 16, wherein the glass comprises particles having a volume-related particle size of from about 0.1 μm to about 10 μm.

27. The composite of claim 25, wherein at least a part of the one or more polymeric materials forms a matrix which comprises at least a part of the particulate glass.

28. The composite of claim 27, wherein at least a part of the one or more polymeric materials forms a sheet-like structure.

29. The composite of claim 28, wherein at least a part of the sheet-like structure is foamed.

30. The composite of claim 1, wherein the one or more polymeric materials are gel-like materials.

31. The composite of claim 1, wherein at least a part of the one or more polymeric materials and of the glass form a layer which has an area weight of from about 400 g/m² to about 1200 g/m².

32. The composite of claim 1, wherein at least a part of the one or more polymeric materials and of the glass form a sheet-like layer having a thickness of from about 0.1 mm to about 2 mm.

33. The composite of claim 1, wherein the one or more polymeric materials have a liquid absorption capacity of at least about 0.5 g/g.

34. The composite of claim 28, wherein the sheet-like structure has a water vapor permeability of from about 100 g/(m²*24 h) to about 5,000 g/(m²*24 h).

35. The composite of claim 34, wherein the water vapor permeability is in the range of from about 300 g/(m²*24 h) to about 1,500 g/(m²*24 h).

36. The composite of claim 1, wherein at least a part of the composite comprises a sheet-like structure and wherein the composite further comprises a backing layer.

37. The composite of claim 36, wherein the backing layer comprises a polymeric material.

38. The composite of claim 37, wherein the polymeric material comprises at least one of a polyurethane and a polyolefin.

39. The composite of claim 37, wherein the backing layer comprises a pressure sensitive adhesive on one side thereof.

40. A wound management article which comprises the composite of claim 1.

41. A bandage which comprises the composite of claim 39.

42. The article of claim 40, which is one of a wound dressing, a wound pad and a compress.

43. The article of claim 40, wherein the article releases the at least one element M at a rate of from about 5 mg/(m²*24 h) to about 50 mg/(m²*24 h).

44. The article of claim 40, wherein the article releases silver at a rate of from about 10 mg/(m²*24 h) to about 40 mg/(m²*24 h).

45. The composite of claim 1, wherein the glass contains from about 0.5% by weight to about 5% by weight of Ag₂O and comprises a particulate glass having a volume-related particle size of from about 0.1 μm to about 10 μm, and wherein the one or more polymeric materials have a liquid absorption capacity of from about 0.5 g/g to about 10 g/g and form a matrix which contains from about 0.1% by weight to about 5% by weight of the glass, based on the combined weight of the glass and the one or more polymeric materials.

46. A method of covering a wound, wherein the method comprises placing in direct contact with the wound at least a portion of a polymeric composite, wherein the composite comprises one or more polymeric materials which are suitable for prolonged direct contact with a wound, at least a part of said at least one or more polymeric materials being associated with a particulate glass comprising an element M which comprises at least one of Ag, Cu, and Zn, which glass, when coming into contact with a wound exudate, is capable of releasing an antimicrobially effective amount of ions of said element M into the wound exudate and comprises about 30-60 mole-% of P₂O₅, about 20-55 mole-% of at least one of CaO and MgO, up to about 5 mole-% of at least one of Na₂O, K₂O and Li₂O, and about 5-50 mole-% of at least one of SiO₂, Al₂O₃ and B₂O₃.

47. The method of claim 46, wherein the one or more polymeric materials have a liquid absorption capacity of at least about 1 g/g.

48. The method of claim 47, wherein the glass comprises particles having a volume-related particle size of from about 0.1 μm to about 10 μm.

49. The method of claim 46, wherein the glass comprises about 45-55 mole-% of P₂O₅, about 40-50 mole-% of at least one of CaO and MgO, up to about 5 mole-% of at least one of Na₂O and K₂O, and about 5-15 mole-% of at least one of SiO₂ and Al₂O₃, and contains from about 0.5% by weight to about 3% by weight of Ag₂O.

50. A process for producing an antimicrobial polymeric composite for use in wound management products, wherein the process comprises combining (a) a particulate glass which comprises about 30-60 mole-% of P₂O₅, about 20-55 mole-% of at least one of CaO and MgO, up to about 5 mole-% of at least one of Na₂O, K₂O and Li₂O, and about 5-50 mole-% of at least one of SiO₂, Al₂O₃ and B₂O₃ and contains an element M which comprises at least one of Ag, Cu, and Zn, which glass, when coming into contact with an aqueous liquid, is capable of releasing ions of said element M into the aqueous liquid, with (b) one or more polymeric materials which are suitable for prolonged direct contact with a wound so that at least a part of the one or more polymeric materials forms a matrix which contains at least a part of the particulate glass.

51. The process of claim 50, wherein the glass is used in an amount of from about 0.01% by weight to about 40% by weight, based on the combined weight of the glass and the one or more polymeric materials.

52. The process of claim 51, wherein the glass comprises from about 0.5% by weight to about 3% by weight of Ag₂O.

53. The process of claim 50, wherein the process further comprises a sterilization of the composite.

54. The process of claim 53, wherein the sterilization comprises irradiating the composite with γ-rays.

55. A polymeric composite for use in wound management products, wherein the composite comprises one or more polymeric materials which are suitable for prolonged direct contact with a wound and are selected from polyacrylates, polyurethanes, styrene butadiene block copolymers, natural rubbers, polysaccharides, heteropolysaccharides and polypeptides, at least a part of said one or more polymeric materials being associated with a glass which comprises an element M which shows antimicrobial activity and comprises at least one of Ag, Au, Pd, Pt, Cu, Ir, Zn, Sn, Sb and Bi, which glass, when coming into contact with an aqueous liquid, is capable of releasing ions of said element M into the aqueous liquid, said glass being present in an amount of from about 0.01% by weight to about 40% by weight, based on the combined weight of the glass and the one or more polymeric materials.

56. The composite of claim 55, wherein the one or more polymeric materials comprise at least one of a polyurethane, a natural rubber, a polysaccharide and a heteropolysaccharide.

57. The composite of claim 55, wherein the one or more polymeric materials comprise a hydrocolloid.

58. The composite of claim 57, wherein the hydrocolloid comprises at least one of microcrystalline cellulose, cellulose, alkylcellulose, hydroxyalkylcellulose, hydroxyalkylalkylcellulose and carboxyalkylcellulose and salts thereof.

59. The composite of claim 55, wherein the one or more polymeric materials comprise at least one of agar agar, carrageen, tragacanth, gum arabic, alginate, pectin, guar flour, carob flour, starch, dextrin, gelatin, xanthan, chitosan and casein.

60. The composite of claim 55, wherein the one or more polymeric materials comprise a hydrogel.

61. The composite of claim 55, wherein the one or more polymeric materials have a white pigment incorporated therein in an amount of from about 0.01% by weight to about 2% by weight, based on the weight of the one or more polymeric materials.

62. The composite of claim 55, wherein the one or more polymeric materials comprise a superabsorber in an amount of from about 0.5% by weight to about 30% by weight, based on the weight of the one or more polymeric materials.

63. The composite of claim 62, wherein the superabsorber comprises units which are derived from acrylic acid.

64. The composite of claim 55, wherein the one or more polymeric materials have incorporated therein a species which comprises at least one of elemental Al, Zn and Mg and basic compounds thereof, the species being present in an amount of from about 0.01% by weight to about 5% by weight, based on the combined weight of the one or more polymeric materials.

65. The composite of claim 55, wherein the glass is present in an amount of from about 0.05% by weight to about 10% by weight, based on the combined weight of the glass and the one or more polymeric materials.

66. The article of claim 56, wherein the glass is present in an amount of from about 0.1% by weight to about 5% by weight, based on the combined weight of the glass and the one or more polymeric materials.

67. The composite of claim 65, wherein the glass comprises not more than about 5% by weight of residual water.

68. The composite of claim 65, wherein the glass comprises a particulate glass.

69. The composite of claim 68, wherein the glass comprises particles having a volume-related particle size of from about 0.1 μm to about 10 μm.

70. The composite of claim 69, wherein the glass comprises about 40-60 mole-% of P₂O₅, about 35-55 mole-% of at least one of CaO and MgO, up to about 5 mole-% of at least one of Na₂O, K₂O and Li₂O, and about 5-20 mole-% of at least one of SiO₂ and Al₂O₃, and contains from about 0.1% by weight to about 10% by weight of at least one of Ag₂O, ZnO and CuO.

71. The composite of claim 65, wherein the glass comprises about 45-55 mole-% of P₂O₅, about 35-50 mole-% of at least one of CaO and MgO, up to about 5 mole-% of at least one of Na₂O and K₂O, and about 5-15 mole-% of at least one of SiO₂ and Al₂O₃, and contains from about 0.5% by weight to about 8% by weight of at least one of Ag₂O and ZnO.

72. The composite of claim 68, wherein the glass comprises about 45-55 mole-% of P₂O₅, about 40-50 mole-% of MgO, and about 5-10 mole-% of Al₂O₃, and contains from about 1% by weight to about 3% by weight of Ag₂O.

73. The composite of claim 55, wherein silver is the only element M contained in the glass and is present therein in an amount of from about 1.5% by weight to about 2.5% by weight of Ag₂O.

74. The composite of claim 55, wherein the composite is capable of releasing silver ions for at least about 48 hours when in contact with a wound exudate.

75. The composite of claim 68, wherein at least a part of the one or more polymeric materials forms a matrix which comprises at least a part of the glass.

76. The composite of claim 55, wherein at least a part of the one or more polymeric materials forms a sheet-like structure.

77. The composite of claim 76, wherein at least a part of the sheet-like structure is foamed.

78. The composite of claim 76, wherein the sheet-like structure has a thickness of from about 0.1 mm to about 2 mm.

79. The composite of claim 78, wherein the sheet-like structure has a water vapor permeability of from about 100 g/(m²*24 h) to about 5,000 g/(m²*24 h).

80. The composite of claim 76, wherein the sheet-like structure has a water vapor permeability of from about 300 g/(m²*24 h) to about 1,500 g/(m²*24 h).

81. The composite of claim 55, wherein at least a part of the one or more polymeric materials and of the glass form a layer which has an area weight of from about 400 g/m² to about 1200 g/m².

82. The composite of claim 55, wherein the one or more polymeric materials have a liquid absorption capacity of at least about 1.5 g/g.

83. The composite of claim 81, wherein the composite further comprises a backing layer.

84. The composite of claim 83, wherein the backing layer comprises a polymeric material.

85. The composite of claim 84, wherein the polymeric material comprises at least one of a polyurethane and a polyolefin.

86. The composite of claim 83, wherein the backing layer comprises a pressure sensitive adhesive on one side thereof.

87. A wound management article which comprises the composite of claim 55.

88. A bandage which comprises the composite of claim 55.

89. The article of claim 87, which is one of a wound dressing, a wound pad and a compress.

90. The article of claim 87, wherein the article releases the at least one element M at a rate of from about 10 mg/(m²*24 h) to about 40 mg/(m²*24 h).

91. A method of covering a wound, wherein the method comprises placing in direct contact with the wound a polymeric composite of claim 55.

92. The method of claim 91, wherein the one or more polymeric materials have a liquid absorption capacity of at least about 1 g/g.

93. The method of claim 92, wherein the glass comprises particles having a volume-related particle size of from about 0.1 μm to about 10 μm.

94. The method of claim 93, wherein the glass comprises about 40-55 mole-% of P₂O₅, about 35-50 mole-% of at least one of CaO and MgO, up to about 5 mole-% of at least one of Na₂O and K₂O, and about 5-20 mole-% of at least one of SiO₂ and Al₂O₃, and contains from about 0.5% by weight to about 5% by weight of Ag₂O.

95. A process for producing an antimicrobial polymeric composite, wherein the process comprises combining (a) one or more polymeric materials which are suitable for prolonged direct contact with a wound and are selected from polyacrylates, polyurethanes, styrene butadiene block copolymers, natural rubbers, polysaccharides, heteropolysaccharides and polypeptides with (b) a glass containing an element M which comprises at least one of Ag, Cu and Zn, which glass, when coming into contact with an aqueous liquid, is capable of releasing ions of said element M into the aqueous liquid, said glass being used in an amount of from about 0.1% by weight to about 10% by weight, based on the combined weight of the glass and the one or more polymeric materials.

96. The process of claim 95, wherein the glass comprises from about 0.5% by weight to about 3% by weight of Ag₂O.

97. The process of claim 96, wherein the process further comprises a sterilization of the composite with γ-rays.

98. The process of claim 97, wherein the sterilization does not result in a noticeable discoloration of the glass.

99. The composite of claim 1, wherein the composite does not show a noticeable discoloration after having been kept at 50° C. for 6 months.

100. The composite of claim 55, wherein the composite does not show a noticeable discoloration after having been kept at 50° C. for 6 months.

101. The composite of claim 1, wherein the composite does not show a noticeable discoloration after having been sterilized with 26 kGy of γ-rays.

102. The composite of claim 1, wherein the composite shows an antimicrobial activity against Escherichia coli IFO 3972 of at least about 3.6, when tested according to JIS 2801:2000.

103. The composite of claim 1, wherein the composite shows an antimicrobial activity against Staphylococcus aureus of at least about 3.3, when tested according to JIS 2801:2000.
